(19) United States
(12) Patent Application Publication
(10) Pub. No.: US 2024/0257984 A1
(43) Pub. Date: Aug. 1, 2024

(54) CLUSTERED ANALYSIS OF TRAINING SCENARIOS FOR ADDRESSING NEURODEVELOPMENTAL DISORDERS

(71) Applicants: Genentech, Inc., South San Francisco, CA (US); Hoffmann-La Roche Inc., Little Falls, NJ (US)

(72) Inventors: Christopher Hughes CHATHAM, San Francisco, CA (US); Matteus Jiawei PAN, San Francisco, CA (US); Katrin Hildegund PRELLER, Zurich (CH); Michael Anthony RABBIA, Nutley, NJ (US); Yajing ZHU, Zurich (CH)

(21) Appl. No.: 18/627,267

(22) Filed: Apr. 4, 2024

Related U.S. Application Data
(63) Continuation of application No. PCT/US22/45818, filed on Oct. 5, 2022.
(60) Provisional application No. 63/252,751, filed on Oct. 6, 2021.

(51) Int. Cl. G16H 50/70; G16H 20/70

(52) U.S. Cl. CPC G16H 50/70; G16H 20/70

(57) ABSTRACT

Methods and systems for simulating training and subpopulations and selecting training scenarios for neurodevelopmental disorders such as autism spectrum disorder (ASD) are described herein. Population data may be received. Skill data indicating skills associated with one or more behaviors exhibited by individuals with one or more neurodevelopmental disorders may be received. Behavioral goals may be identified. Scenario data indicating a plurality of different training scenarios may be generated. Efficacy data may be generated by estimating a probability that skills would be trained by a given training scenario. Estimated clinical success data may be generated by simulating, for each training scenario and for each subject subpopulation, a degree of behavioral change. A combination of a first subject subpopulation and a first training scenario may be selected. The first training scenario may be associated with training a plurality of different skills.

### CROSS-REFERENCE TO RELATED APPLICATIONS

This application is a continuation of International Application No. PCT/US2022/045818, filed on Oct. 5, 2022, which claims the benefit of and the priority to U.S. Provisional Application No. 63/252,751, entitled “CLUSTERED ANALYSIS OF TRAINING SCENARIOS FOR ADDRESSING NEURODEVELOPMENTAL DISORDERS” and filed on Oct. 6, 2021, which are hereby incorporated by reference in their entirety for all purposes.

### TECHNICAL FIELD

Aspects described herein generally relate to medical treatment and medical devices for improved subject testing and analysis.

### BACKGROUND

Neurodevelopmental disorders (NDs), such as autism spectrum disorder (ASD), encompass a broad range of conditions which may negatively impair an individual's social, communicative, and/or behavioral capabilities. Individuals experiencing one or more NDs may experience difficulty communicating and interacting with others, may have particularly restricted interests, and/or may exhibit repetitive behaviors. For example, daily life tasks involving social interaction may present a particular difficulty for an individual experiencing one or more NDs. NDs are often accompanied by sensory sensitivities and medical issues such as gastrointestinal disorders, seizures or sleep disorders, as well as mental health challenges such as anxiety, depression and attention issues. As such, individuals experiencing one or more NDs may have difficulties at school, at work, and in other social contexts.

Various treatment methods exist for various NDs. Generally, early identification of symptoms associated with NDs in children is valuable, as early intervention strategies (e.g., therapy to help children experiencing one or more NDs talk, walk, and generally otherwise interact with others) can be beneficial. Applied Behavior Analysis (ABA), a common approach, entails encouraging positive behaviors (e.g., social interaction) and discouraging negative behaviors (e.g., being withdrawn or non-communicative). Within the category of ABA, a number of approaches exist for NDs such as ASD, including discrete trial training (e.g., testing and rewarding positive behavior in discrete tasks), early intensive behavioral intervention, pivotal response training (e.g., encouraging a subject to learn monitor their own behavior), verbal behavior intervention, occupational therapy (e.g., helping the subject live independently by learning to dress, eat, bathe, and perform other tasks), sensory integration therapy (e.g., helping the subject handle unwelcome sights, sounds, and smells), and the like. Other approaches include modifying the individual's diet, using medication, and the like.

The voluminous different ways in which individuals experiencing one or more NDs may be provided treatment, as well as the various different subpopulations with one or more NDs that experience those one or more NDs differently, can make the task of treating individuals with one or more NDs particularly difficult. Different subpopulations may react to different forms of treatment differently, even though it is often more efficient (and cost-effective) to implement similar forms of therapy to groups of individuals. For example, it may be possible to provide treatment to a group of individuals experiencing one or more NDs, but clinicians may have difficulty identifying which forms of treatment should be provided, let alone which forms of treatment may be maximally effective for the particular configuration of individuals in the group. This is particularly the case when treatment involves the training of multiple skills (e.g., tasks which require multiple life skills, such as looking individuals in the eyes, speaking out loud, providing money to a cashier, etc.) which impact multiple dimensions of one or more NDs.

### SUMMARY

The following presents a simplified summary of various aspects described herein. This summary is not an extensive overview and is not intended to identify required or critical elements or to delineate the scope of the claims. The following summary merely presents some concepts in a simplified form as an introductory prelude to the more detailed description provided below.

To overcome limitations in the prior art described above, and to overcome other limitations that will be apparent upon reading and understanding the present specification, aspects described herein are directed towards simulating various training scenarios and various subject subpopulations to ascertain degrees of behavioral change by subject subpopulations, then selecting combinations of subject subpopulations and training scenarios that beneficially train different skills associated with one or more NDs.

A computing device may be configured to receive population data that indicates a plurality of different subject subpopulations. The computing device may receive skill data that indicates a plurality of different skills associated with one or more behaviors exhibited by individuals experiencing one or more NDs. The computing device may identify, based on the population data and the skill data, behavioral goals for each subject subpopulation of the plurality of different subject subpopulation. Those behavioral goals may relate to the improvement of one or more commonly-unattained skills for each subject subpopulation of the plurality of different subject subpopulation. The computing device may generate scenario data 152 which indicates a plurality of different training scenarios for training one or more skills of the plurality of different skills. The computing device may generate efficacy data by estimating, for each training scenario of the plurality of different training scenarios, a probability that each skill of the plurality of different skills can be trained by the training scenario. The computing device may generate estimated clinical success data by simulating, for each training scenario of the plurality of different training scenarios and for each subject subpopulation of the plurality of different subject subpopulation, a degree of behavioral change by the subject subpopulation. The computing device may then select, based on the behavioral goals, the efficacy data, and the estimated clinical success data, a combination of a first subject subpopulation of the plurality of different subject subpopulations and a first training scenario of the plurality of different training scenarios. The first training scenario of the plurality of different training scenarios may be associated with training two or more of the plurality of different skills.

As will be described in greater detail below, the computing device may be configured in a variety of ways. The computing device may cause an extended reality device to provide, to a user associated with the first subject subpopulation, an extended reality environment based on the first training scenario. The computing device may select the combination by identifying that at least one of the different subject subpopulations has not performed a training scenario associated with the two or more of the plurality of different skills. The computing device may generate the estimated clinical success data using standards associated with one or more NDs, such as one or more of the Vineland Adaptive Behavior Scale (VABS), or a Goal Attainment Scale (GAS). The computing device may select the combination based on a trainability value assigned to the two or more of the plurality of different skills. The computing device may simulate the degree of behavioral change by the subject subpopulation using the Monte Carlo method to simulate a performance level of each subject subpopulation of the plurality of different subject subpopulations. The computing device may select the combination by selecting at least two subject subpopulations of the plurality of different subject subpopulations. The computing device may transmit, by to a user computing device, an indication of the combination. In that circumstance, transmitting the indication of the combination may cause the user computing device to display the indication of the combination. The estimated clinical success data may indicate one or more of: an absolute effect size of a performance level of the subject subpopulation, or a standardized effect size of the performance level of the subject subpopulation. The computing device may simulate the degree of behavioral change by weighting the performance level by applying a function to the performance level. In that circumstance, the function may be based on a rarity of each subject subpopulation of the plurality of different subject subpopulations. The computing device may identify the behavioral goals based on one or more of: a range of subject ages, a range of Full-Scale Intelligence Quotient (FSIQ) values, or a range of Social Responsiveness Scale—Version 2 (SRS Total) t-scores.

These and additional aspects will be appreciated with the benefit of the disclosures discussed in further detail below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

A more complete understanding of aspects described herein and the advantages thereof may be acquired by referring to the following description in consideration of the accompanying drawings, in which like reference numbers indicate like features, and wherein:

FIG. 1 depicts an illustrative computer system architecture that may be used in accordance with one or more illustrative aspects described herein.

FIG. 2 depicts an illustrative flowchart with steps that may be performed by a computing device to determine combinations of therapy and target population.

FIG. 3 depicts a first message diagram between databases, a computing device, and input/output.

FIG. 4 depicts a second message diagram between databases, a computing device, and input/output.

FIG. 5 depicts a first message diagram between the databases, various elements of a computing device, and input/output.

FIG. 6 depicts a second message diagram between the databases, various elements of a computing device, and input/output.

FIG. 7 depicts an example of commonly-unattained skills for a subject subpopulation.

FIG. 8 depicts illustrative correlations between different subpopulations and commonly-unattained skills.

FIG. 9 depicts examples of probabilities that skills may be improved for a subject subpopulation via a training scenario.

FIG. 10 depicts an example heat map representing associations between various unattained skills for a subject subpopulation.

### DETAILED DESCRIPTION

In the following description of the various embodiments, reference is made to the accompanying drawings identified above and which form a part hereof, and in which is shown by way of illustration various embodiments in which aspects described herein may be practiced. It is to be understood that other embodiments may be utilized, and structural and functional modifications may be made without departing from the scope described herein. Various aspects are capable of other embodiments and of being practiced or being carried out in various different ways.

As a general introduction to the subject matter described in more detail below, aspects described herein are directed towards treating one or more symptoms of social, communicative, and/or sensory deficits in an individual experiencing one or more NDs. Individuals experiencing one or more NDs may be trained using training scenarios, such as by simulating a life task (e.g., purchasing goods at a convenience store). Such training scenarios may be configured to train skills associated with one or more behaviors exhibited by individuals with one or more NDs. For example, a training scenario involving purchasing goods at a convenience store may entail training an individual to practice speaking to a cashier, looking the cashier in the eyes, use appropriate body language during a transaction, and the like. That said, different subject subpopulations may have different levels of proficiency with certain skills, and different training scenarios may impact different subject subpopulations differently. For example, younger individuals experiencing one or more NDs may have more difficulty making eye contact than older individuals, such that training scenarios involving eye contact may be more difficult for younger subject subpopulations as compared to older subject subpopulations. Clinicians may detect isolated instances of patient difficulty, but generally have limited insight into such difficulty in the aggregate, particularly when multiple subpopulations are trained in the same training scenario. Aspects described herein remediate the above-identified issues, among others, by performing particularized processing steps in view of the unique needs of one or more NDs to identify combinations of subject subpopulations and training scenarios which may have maximal benefit for the development of skills unattained by those subject subpopulations. In other words, aspects described herein use unique simulation strategies and processing techniques to identify unforeseen ways in which one or more NDs may be treated in subject subpopulations.

Though Autism Spectrum Disorder is referenced throughout the present disclosure as one example of a neurodevelopmental disorder, the present disclosure is not limited to autism spectrum disorder. Similarly, the term neurodevelopmental disorder is not intended to refer to a particular definition of a neurodevelopmental disorder, such as might be provided by various versions of the Diagnostic and Statistical Manual of Mental Disorders (DSM). Rather, the present disclosure may be freely applied to a wide variety of neurodevelopmental disorders. Indeed, the present disclosure may be beneficially applied to train subjects experiencing one or more neurodevelopmental disorders, whether or not those one or more neurodevelopmental disorders have a phenotype consistent with autism spectrum disorder. For example, the improvements described herein may be used to help train individuals experiencing various neuromuscular conditions which inhibit fine and gross motor skills.

Moreover, the present disclosure may be applied to caregivers for those experiencing neurodevelopmental disorders. In other words, while much of this disclosure focuses on the training of individuals experiencing one or more neurodevelopmental disorders for the sake of ease of explanation, the same process may be applied to train individuals who help provide support to (e.g., are caregivers of) other individuals experiencing one or more neurodevelopmental disorders. For example, the present disclosure may advantageously train caregivers to improve skills associated with the care of individuals experiencing autism spectrum disorder.

Aspects detailed herein improve the functioning of computers by providing a method to process data specific to one or more NDs to simulate training scenarios and identify unique combinations of skills and subject subpopulations that may be trained using real-life training scenarios. The processing and simulation steps described herein are specific to unique aspects of one or more NDs and reflect the fact that different subject subpopulations may have different proficiency with ordinary life skills (e.g., speaking out loud). The processing and simulation techniques described herein could not be performed by a human being, whether or not with pen and paper: the data is so voluminous as to be completely infeasible for human processing, and the simulation and processing steps are necessarily computer-implemented.

It is to be understood that the phraseology and terminology used herein are for the purpose of description and should not be regarded as limiting. Rather, the phrases and terms used herein are to be given their broadest interpretation and meaning. The use of “including” and “comprising” and variations thereof is meant to encompass the items listed thereafter and equivalents thereof as well as additional items and equivalents thereof. The use of the terms “connected” and similar terms, is meant to include both direct and indirect connecting.

### Computing Environment

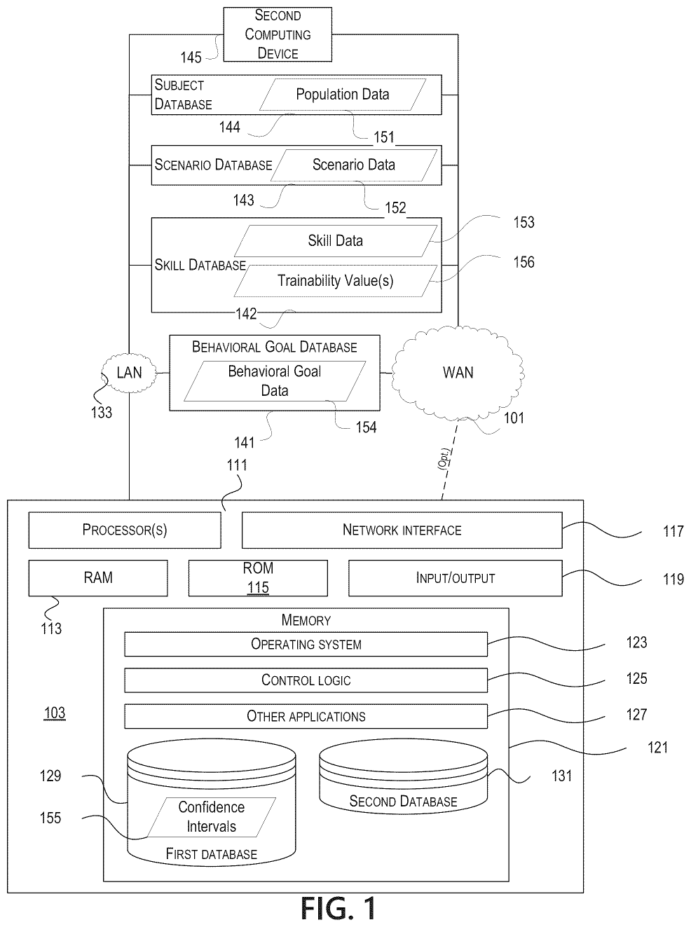

FIG. 1 illustrates one example of a system architecture and data processing device that may be used to implement one or more illustrative aspects described herein in a standalone and/or networked environment. A computing device 103 may be interconnected via a wide area network (WAN) 101, such as the Internet. Other networks may also or alternatively be used, including private intranets, corporate networks, local area networks (LAN), metropolitan area networks (MAN), wireless networks, personal networks (PAN), and the like. The network 101 is for illustration purposes and may be replaced with fewer or additional computer networks. A local area network 133 may have one or more of any known LAN topologies and may use one or more of a variety of different protocols, such as Ethernet. Computing devices, such as the computing device 103, a second computing device 145, a subject database 144, a scenario database 143, a skill database 142, a behavioral target database 141, and/or other devices (not shown) may be connected to one or more of the networks via twisted pair wires, coaxial cable, fiber optics, radio waves, or other communication media.

The term “network” as used herein and depicted in the drawings refers not only to systems in which remote storage devices are coupled together via one or more communication paths, but also to stand-alone devices that may be coupled, from time to time, to such systems that have storage capability. Consequently, the term “network” includes not only a “physical network” but also a “content network,” which is comprised of the data—attributable to a single entity—which resides across all physical networks.

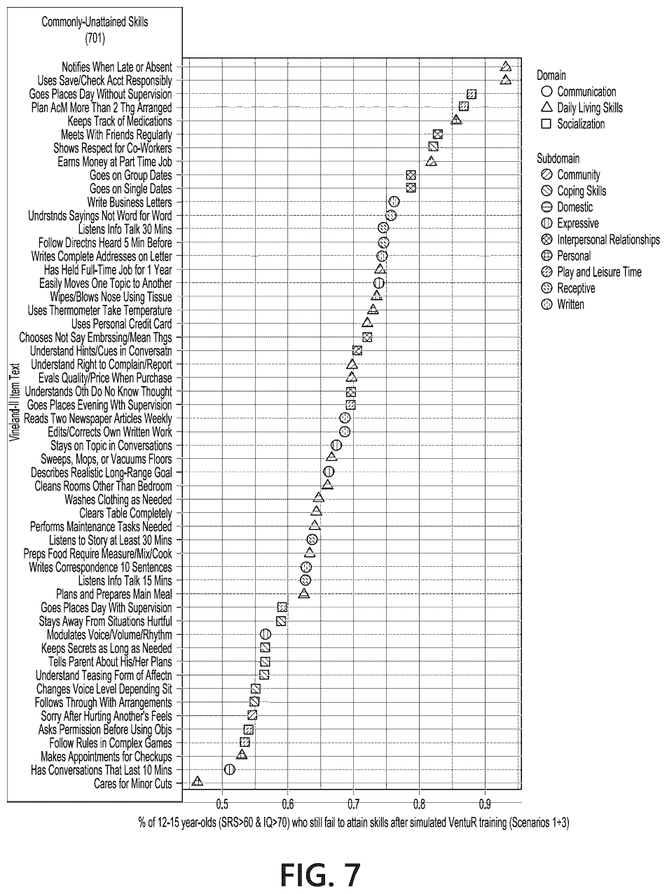

The skill database 142 may store skill data 153 that indicates plurality of different skills associated with one or more behaviors exhibited by individuals with one or more NDs. A skill may be any task (e.g., life skill, ability, or the like) which may be associated with a subject. For example, a skill may relate to a subject's ability to write, their ability to handle domestic tasks, their ability to cope with change or negative experiences, their hygiene, or the like. The skills may correspond to various domains, such as communication skills (e.g., verbal speaking), daily living skills (e.g., making a bed after waking up), and/or socialization skills (e.g., making friends). Additionally and/or alternatively, the skills may relate to subdomains such as community skills (e.g., participating in group activities), coping skills (e.g., dealing with negative experiences), domestic skills (e.g., cleaning their room), expressive skills (e.g., expressing emotions), interpersonal relationship skills (e.g., making and keeping friends), personal skills (e.g., taking showers), play and leisure time skills (e.g., sharing toys), receptive skills (e.g., understanding others' emotions), and/or written skills (e.g., writing e-mails). The skill data 153 may be represented as a list of skills, such as an ordered list of skills separated into various domains and/or subdomains. Examples of such skills are listed in greater detail below with respect to FIG. 7.

The skill database 142 may additionally and/or alternatively store trainability values 156. A trainability value, such as one or more of the trainability values 156, may represent the ability of one or more skills to be taught to a subject. For example, it may be reasonably straightforward to (with time and effort) teach subjects to write, but it may be somewhat more difficult to teach the same individuals to make and keep long-term friends. As such, while writing skills may be provided a trainability value that indicates training is moderately easy, maintaining-friendship skills may be provided a trainability value that indicates training is somewhat more difficult.

The subject database 144 may store population data 151 that provides information relating to a plurality of different subject subpopulations. A subject subpopulation may be any division of a population of subjects experiencing one or more NDs. For example, subpopulations may be based on demographics such as age, sex, location, income level, or the like. In this manner, for instance, one subject subpopulation may correspond to children aged two to eight years old, while another subject subpopulation may correspond to children aged nine through twelve years old. As another example, one subject subpopulation may correspond to adults in New York, whereas another subject subpopulation may correspond to adults in California. The population data 151 may be on a per-subject level. For example, the population data 151 may indicate, for each of a plurality of different subjects, corresponding demographic information.

The subject database 144 may additionally and/or alternatively store historical simulation information for one or more subjects. For example, the historical simulation information may indicate whether certain subjects have been provided certain training scenarios. Additionally and/or alternatively, the historical simulation information may comprise indications of whether certain subjects have passed certain diagnostic tests provided during training scenarios.

The subject database 144 may additionally and/or alternatively store proficiency information for one or more subjects. Such proficiency information may comprise information relating to the ability of one or more subjects to perform skills. For example, the population data 151 may comprise, for a particular subject, an indication of scores, associated with one or more skills, provided to that particular subject during clinical testing.

The behavioral target database 141 may comprise behavioral goal data 154 indicating data such as one or more commonly-unattained skills for each subject subpopulation of the plurality of different subject subpopulation. Examples of such commonly-unattained skills are provided in FIG. 7 as the commonly-unattained skills 701. Such behavioral goal data 154 may be identified (e.g., generated) based on the population data 151 and/or the skill data 153. For example, the behavioral goal data 154 may be identified based on the population data 151 and/or the skill data 153, such that the behavioral goals may correspond to the improvement of one or more commonly-unattained skills 701 for each subject subpopulation of the plurality of different subject subpopulation. In this manner, the behavioral goal data 154 may indicate skills that one or more subject subpopulations need to improve. For example, the behavioral goal data 154 may indicate that boys aged eight to eleven that experience one or more NDs have difficulty regularly taking showers, whereas girls aged twelve to fifteen that experience one or more NDs have difficulty speaking out loud. Such behavioral goal data 154 may be represented as data that indicates, for one or more subject subpopulations, scores (e.g., subjective or objective scores) corresponding to one or more skills. For example, for boys aged eight to eleven that experience one or more NDs, they may be scored “good” on skills involving making friends, but “bad” on skills involving writing skills, suggesting that training scenarios that help improve writing skills may be worthwhile.

The scenario database 143 may indicate one or more different training scenarios for training one or more skills associated with one or more NDs. A training scenario may be any activity which may be used to train (e.g., improve the performance of) one or more skills associated with one or more NDs. For training scenarios targeting skills in the domestic subdomain, for example, the training scenarios may focus on training an individual to clean their home. For training scenarios targeting skills in the expressive subdomain, for example, the training scenarios may focus on training an individual to speak out loud. For training scenarios targeting skills in the receptive subdomain, for example, the training scenarios may focus on training an individual to identify facial expressions. The training scenarios might be all or portions of an interactive application (e.g., a game) which might be provided to a subject subpopulation. For example, the scenario database 143 might store software modules which might be used to provide, via a virtual reality, augmented reality, and/or mixed reality interface, a training scenario.

With respect to providing scenarios via a virtual reality, augmented reality, and/or mixed reality interface, training scenarios might be provided in accordance with the techniques described in International Patent Application No. PCT/US2020/065805, which is incorporated by reference herein.

Training scenarios may train a plurality of different skills, including skills in different domains and/or subdomains. Indeed, such training scenarios may be quite efficient in that they might address multiple dimensions of issues experienced by those experiencing one or more NDs. For example, a training scenario may involve a subject purchasing an item from a convenience store. Such a training scenario may involve expressive subdomain skills (e.g., speaking out loud to a cashier), domestic subdomain skills (e.g., paying for items with cash or a credit card), and receptive subdomain skills (e.g., understanding what a cashier says and does and reacting appropriately). Training scenarios may additionally and/or alternatively involve multiple subjects, including subjects from different subject subpopulations. For example, in the convenience store training scenario referenced above, one subject may play the role of the cashier, and another subject may play the role of a purchaser.

The second computing device 145 may be a computing device associated with a clinician, a subject, or the like. As will be described further below, recommendations for training scenarios may be output to computing devices, such as the second computing device 145. Such a recommendation may be displayed on, e.g., a clinician's computer, and/or may be output for a subject (e.g., to prompt them to voluntarily participate in one or more training scenarios). The recommendation may additionally and/or alternatively be used to automatically initiate a training scenario. For example, in circumstances where a training scenario may be able to be performed using a smartphone (e.g., as part of a gamified training scenario, and/or as part of a call which may be initiated by the smartphone), the recommendation may be used by the smartphone (e.g., the second computing device 145) to initiate the training scenario. As another example, the second computing device 145 may be a virtual reality, augmented reality, and/or mixed reality headset.

Computing devices, including applications executing thereon, may be combined on the same physical machines, and retain separate virtual or logical addresses, or may reside on separate physical machines. FIG. 1 illustrates just one example of a network architecture that may be used, and those of skill in the art will appreciate that the specific network architecture and data processing devices used may vary, and are secondary to the functionality that they provide, as further described herein. For example, services provided by skill database 142 and the subject database 144 may be combined on a single computing device.

Computing devices, such as the computing device 103, the behavioral target database 141, the skill database 142, the scenario database 143, the subject database 144, and/or the second computing device 145, may be any type of known computer, server, or data processing device. The computing device 103, for example, may include one or more processors 111 controlling overall operation of the computing device 103. The computing device 103 may further include random access memory (RAM) 113, read only memory (ROM) 115, a network interface 117, input/output interfaces 119 (e.g., keyboard, mouse, display, printer, etc.), and/or memory 121. The input/output (I/O) 119 may include a variety of interface units and drives for reading, writing, displaying, and/or printing data or files. The memory 121 may further store operating system software 123 for controlling overall operation of the computing device 103, control logic 125 for instructing the computing device 103 to perform aspects described herein, and other application software 127 providing secondary, support, and/or other functionality which may or might not be used in conjunction with aspects described herein. The control logic 125 may also be referred to herein as the software 125. Functionality of the software 125 may refer to operations or decisions made automatically based on rules coded into the control logic 125, made manually by a user providing input into the system, and/or a combination of automatic processing based on user input (e.g., queries, data updates, etc.).

The memory 121 may also store data used in performance of one or more aspects described herein, including a first database 129 and a second database 131. The first database 129 may include the second database 131 (e.g., as a separate table, report, etc.). The first database 129 may store data, such as confidence intervals 155, that may be used for the purposes of simulating training scenarios. That is, the information can be stored in a single database, or separated into different logical, virtual, or physical databases, depending on system design. Computing devices, such as the behavioral target database 141, may have similar or different architecture as described with respect to the computing device 103. Those of skill in the art will appreciate that the functionality of the computing device 103 (or any other computing device described herein) as described herein may be spread across multiple data processing devices, for example, to distribute processing load across multiple computers, to segregate transactions based on geographic location, user access level, quality of service (QoS), etc.

One or more aspects may be embodied in computer-usable or readable data and/or computer-executable instructions, such as in one or more program modules, executed by one or more computers or other devices as described herein. Generally, program modules include routines, programs, objects, components, data structures, etc. that perform particular tasks or implement particular abstract data types when executed by a processor in a computer or other device. The modules may be written in a source code programming language that is subsequently compiled for execution or may be written in a scripting language such as (but not limited to) HyperText Markup Language (HTML) or Extensible Markup Language (XML). The computer executable instructions may be stored on a computer readable medium such as a nonvolatile storage device. Any suitable computer readable storage media may be utilized, including hard disks, CD-ROMs, optical storage devices, magnetic storage devices, and/or any combination thereof. In addition, various transmission (non-storage) media representing data or events as described herein may be transferred between a source and a destination in the form of electromagnetic waves traveling through signal-conducting media such as metal wires, optical fibers, and/or wireless transmission media (e.g., air and/or space). Various aspects described herein may be embodied as a method, a data processing system, or a computer program product. Therefore, various functionalities may be embodied in whole or in part in software, firmware, and/or hardware or hardware equivalents such as integrated circuits, field programmable gate arrays (FPGA), and the like. Particular data structures may be used to more effectively implement one or more aspects described herein, and such data structures are contemplated within the scope of computer executable instructions and computer-usable data described herein.

### Neurodevelopmental Disorder Training Scenario Simulation

Discussion will now turn to selection of combinations of training scenarios and subject subpopulations based on simulation of degrees of behavioral change.

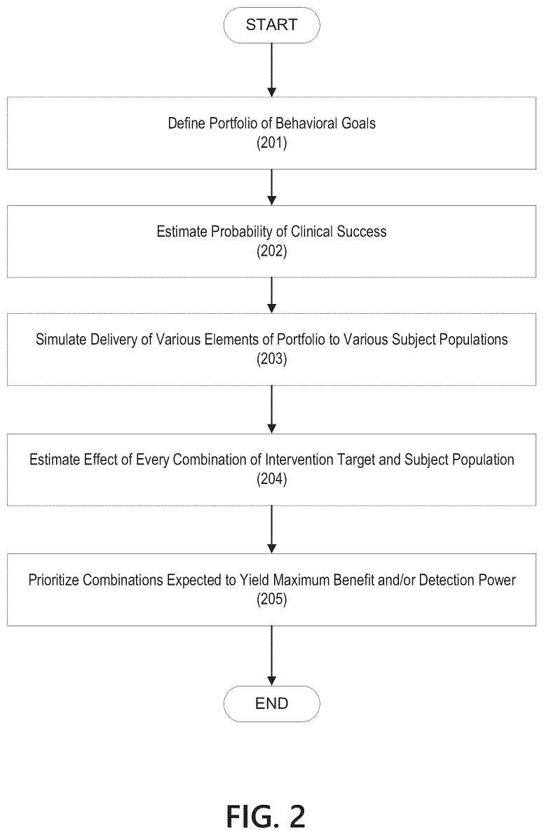

FIG. 2 depicts a flow chart of a method which may be performed by a computing device to select combinations of training scenarios and subject subpopulations. A computing device may comprise one or more processors and memory storing instructions that, when executed by the one or more processors, cause performance of one or more of the steps of FIG. 2. Additionally and/or alternatively, one or more non-transitory computer-readable media may store instructions that, when executed by a computing device, cause performance of one or more of the steps of FIG. 2. The steps depicted in FIG. 2 are illustrative and may be rearranged or otherwise modified as desired. For example, multiple steps may be performed in between step 201 and step 202, and/or step 205 may be replaced and/or omitted.

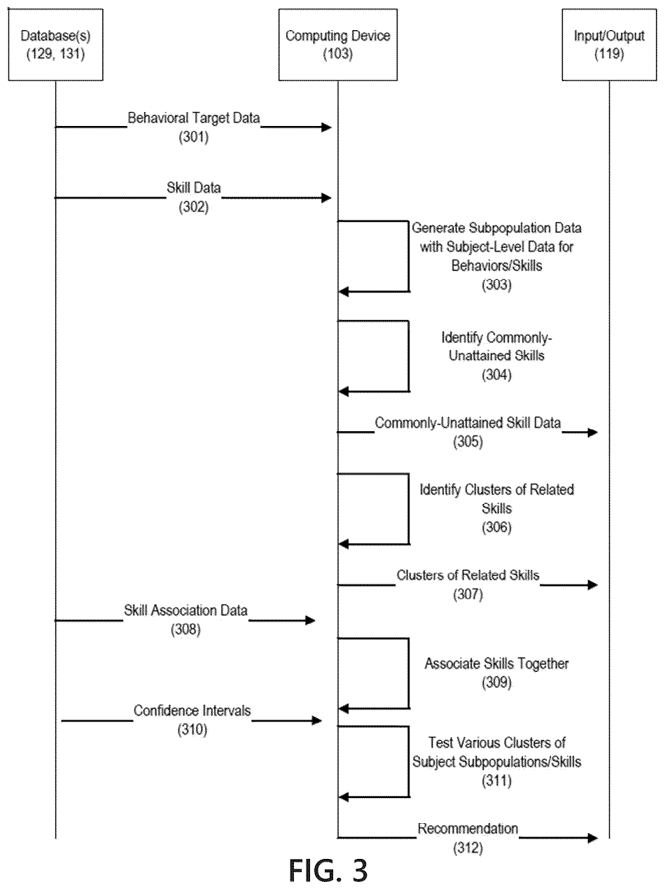

As an introduction to FIG. 2, the processes described herein many advantageously simulate combinations of intervention targets (e.g., skills to be learned) and subject populations. In this manner, a computing device may thereby use simulation and processing techniques to identify unique opportunities to address aspects of one or more NDs by combining one or more subject subpopulations and one or more training scenarios (which, themselves, address one or more skills associated with one or more NDs). FIG. 2 represents a high-level manner in which such simulation may be performed. Other figures discussed below (e.g., FIGS. 3-6) provide examples of similar processes in much greater detail.

In step 201, the computing device may define a portfolio of behavioral goals. Such behavioral goals may be stored by the behavioral target database 141 and/or may correspond to commonly-unattained skills 701, such as those which may be reflected by the subject database 144 and/or the skill database 142. The behavioral goals may additionally and/or alternatively relate to targets for individuals experiencing one or more NDs. The behavioral goals may additionally and/or alternatively correspond to various training scenarios, such as ways in which those commonly-unattained skills 701 may be trained. For example, the behavioral goals may correspond to verbal communication, and known training scenarios for verbal communication may include speech practice.

In step 202, the computing device may estimate a probability of clinical success. For each skill identified in step 201, the computing device may determine (e.g., predict, estimate, and/or ascertain) a probability that such a skill may be trained (e.g., improved) by one or more training scenarios. For example, for communication domain skills, speech practice training scenarios may be particularly effective, whereas training scenarios that only incidentally involve speech (e.g., training scenarios involving shopping at a store) may be minimally effective. In this manner, the computing device may determine a likelihood that any given training scenario will train (e.g., improve) a particular skill.

In step 203, the computing device may simulate delivery of various elements of the portfolio to various subject populations. In this simulation process, the computing device may iterate through various combinations of training scenarios and subject subpopulations. This process may be performed based on historical, real-life testing: for example, the simulation process may be based on data collected from real training scenarios performed on real subjects.

In step 204, the computing device may estimate the effect of every combination of an intervention target and a target population. Based on the simulation(s) performed in step 203, the computing device may ascertain an effectiveness of training scenarios with respect to various subject populations. For example, the computing device may generate, for each and every possible combination of one or more subject subpopulations and one or more training scenarios, an efficacy of the combination with respect to one or more skills associated with one or more NDs. This efficacy may be reflected in any objective and/or subjective measurement: for example, the efficacy may be “high” if a simulated training scenario is predicted to significantly improve the performance of commonly-unattained skills 701 for a particular subject subpopulation, whereas the efficacy may be “low” if a simulated training scenario is predicted to improve the performance of commonly-unattained skills 701 only somewhat for a particular subject subpopulation.

The process described in step 204 may be based on historical reports of efficacy of various forms of training. For example, the computing device may receive information from users (e.g., subjects experiencing one or more neurodevelopmental disorders, therapists, or the like) regarding change(s) in skills and/or other subject characteristics. Such information may indicate the efficacy of various training scenarios on various skills. In this manner, the estimated efficacy in step 204 may be based on historical real-world testing of one or more combinations of intervention target(s) and target population(s).

In step 205, the computing device may prioritize combinations of intervention target (e.g., training scenarios) and target population (e.g., subject subpopulation(s)) that are expected to yield a maximum benefit and/or detection power. In this manner, the computing device may, based on the efficacies determined in step 204, identify one or more particularly valuable combinations of one or more subject subpopulations and one or more training scenarios to try in real life. Such a step may, in practice, involve causing one or more training scenarios to occur in real life, in a virtual, augmented, and/or mixed reality environment, in a software application, or the like. For example, if the estimated effect of a particular combination (as determined in step 204) is particularly high, then the computing device may transmit a message to a smartphone application on the subject(s) smartphones (e.g., the second computing device 145) so as to initiate the beginning of this training scenario. As a more particular example, if the process depicted in step 204 indicates that adult males aged twenty-five to thirty experiencing one or more NDs would be particularly improved in the communication domain if they were prompted to go out on a speed dating event, then the computing device may transmit, to smartphones associated with adult males aged twenty-five to thirty experiencing one or more NDs, a message prompting the subjects to attend a local speed dating event, including a calendar invite for a recent speed dating event.

FIG. 3 depicts a messaging diagram between databases, the computing device 103, and input/output 119. The devices depicted in FIG. 3 are illustrative and may be rearranged as desired. For example, the databases may include, for example, the behavioral target database 141, the skill database 142, the scenario database 143, and/or the subject database 144. The messages depicted in FIG. 3 are illustrative, and may be rearranged, omitted, and/or revised as desired.

In step 301, the computing device 103 may receive behavioral goal data 154 from databases such as the first database 129, the second database 131, and/or the behavioral target database 141. As indicated above with respect to the behavioral target database 141, the behavioral goal data 154 may indicate targets (e.g., goals) for individuals with one or more NDs and/or one or more training scenarios to address those targets. For example, the behavioral goal data 154 may indicate one or more skills that are commonly-unattained by individuals experiencing one or more NDs and which sorts of training scenarios may be used to train those skills. In this manner, the behavioral goal data 154 may comprise, for example, a listing of various training scenarios (e.g., those stored by the scenario database 143) which may be performed by subjects, with those training scenarios indicating one or more skills (and/or skill domains/subdomains) which are believed to be improved by those training scenarios. For example, the behavioral goal data 154 may indicate that individuals experiencing one or more NDs often have difficulty with verbal communication, and may indicate one or more training scenarios (e.g., stored by the skill database 142) which may be used to train verbal communication skills, with each of the one or more training scenarios weighted based on their efficacy with respect to training verbal communication skills.

In step 302, the computing device 103 may receive skill data 153 from databases such as the first database 129, the second database 131, and/or the skill database 142. As indicated with respect to FIG. 1, the skill data 153 may indicate a plurality of different skills associated with one or more behaviors exhibited by individuals experiencing one or more NDs. This data may divide the skills into various domains and/or subdomains. For example, step 302 may comprise the computing device 103 receiving a listing of various skills, with such skills grouped into various domains and/or subdomains.

In step 303, the computing device 103 may generate subpopulation data 151 with subject-level data for behaviors and/or skills. The computing device may thereby determine which subject subpopulations exist and/or which skills and/or behaviors those subject subpopulations excel at and/or have difficulty with. As part of this process, the computing device may receive subject data from the subject database 144. Such subject data may comprise information about one or more subjects, such as past training scenarios performed by one or more of the subjects, evaluations (e.g., diagnostic tests) of the one or more subjects, or the like. Using the subject data, the computing device 103 may generate data indicating, for one or more subjects, data indicating performance of those subjects with respect to various skills, skill domains, and/or skill subdomains. For example, the computing device 103 may process subject data from the subject database 144 to determine, for each subject, how well the subject communicates verbally, then aggregate these determinations for various subject subpopulations. The computing device 103 may group the subject data into subpopulations based on demographics such as age, sex, location, income level, or the like. In this manner, the computing device 103 may generate data indicating, for one or more subject subpopulations, information about subjects within the subpopulation.

In step 304, the computing device 103 may identify commonly-unattained skills (e.g., the commonly-unattained skills 701) for one or more subjects and/or one or more subject subpopulations. Based on the subpopulation data 151 generated in step 303, the computing device 103 may identify one or more skills that are commonly-unattained by one or more subjects and/or subject subpopulations. In this manner, the computing device 103 may identify skill deficiencies across multiple subjects. For example, males aged twelve to fifteen experiencing one or more NDs may commonly have issues with verbal communication skills, whereas girls aged twelve to fifteen experiencing one or more NDs may commonly have issues with writing communication skills.

In step 305, the computing device 103 may output (e.g., via the input/output 119) skill data 153 which may indicate the identified commonly-unattained skills referenced in step 304. Such output may be stored in a database (e.g., the first database 129 and/or the second database 131. Such skill data 153 may be particularly valuable for the purposes of tracking and research. For example, the output of the skill data 153 may comprise causing display of the skill data 153 in a user interface, such that a researcher may analyze trends in the data.

In step 306, the computing device 103 may identify clusters of related skills. A cluster may be any grouping of two or more skills based on, e.g., similarities or relations between those skills. The computing device 103 may identify, based on the skill data 153, clusters of commonly-unattained skills (e.g., the commonly-unattained skills 701) across one or more subpopulations. For example, a particular subject subpopulation may be deficient in numerous skills in a particular skill domain or skill subdomain, such as a verbal communication subdomain. As another example, two closely-related subject subpopulations (e.g., subpopulations nearby in age, such as a first subpopulation of males aged twelve to fifteen and a second subpopulation of males aged sixteen to eighteen) may both have difficulty with numerous skills in the written communication subdomain.

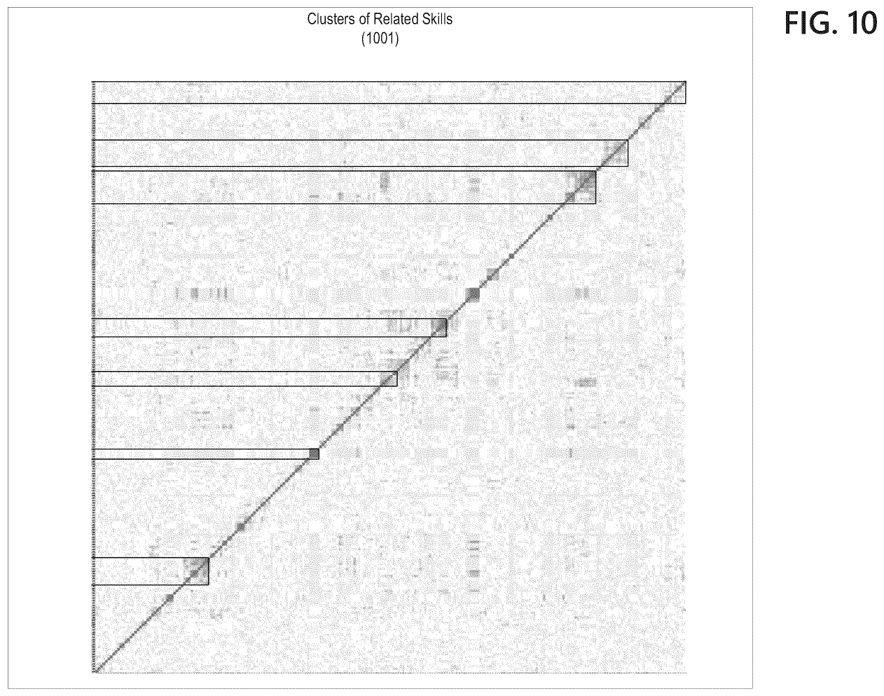

In step 307, the computing device 103 may output (e.g., via the input/output 119) the clusters of related skills (e.g., clusters of related skills 1001 as shown in FIG. 10). As with the skill data 153, these clusters of related skills 1001 may be themselves particularly valuable for the purposes of research. For example, the output of the clusters of related skills 1001 may comprise causing display of the clusters in a user interface, such that a researcher may analyze trends in the data.

The output of clusters of related skills may include longitudinal data on those skills, including changes in skills or other subject characteristics over time. For example, the output clusters might indicate a predicted change in the clusters over time, and/or one or more indications of how the related skills may be related.

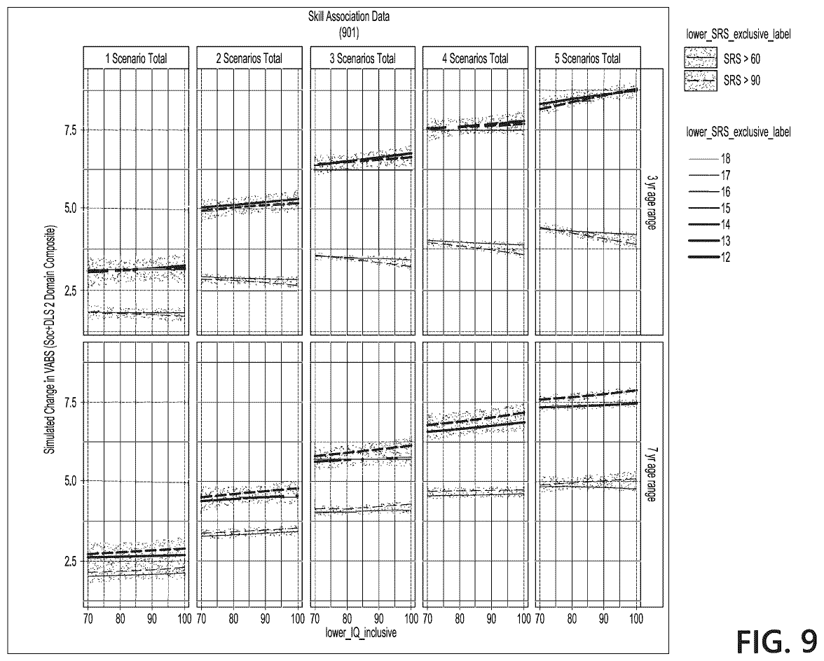

In step 308, the computing device 103 may receive skill association data from databases such as the first database 129, the second database 131, and/or the skill database 142. Skill association data (e.g., as shown in FIG. 9 as skill association data 901) may indicate one or more associations between different skills. For example, the skill association data 901 may indicate two or more skills that may be trained together as part of the same training scenario. As another example, the skill association data 901 may indicate that one skill tends to improve when another skill is trained. As such, as compared to the clusters identified in step 306, the skill association data 901 might reflect third party testing, research, or the like, such that it might originate from databases external to the computing device 103. Such information may be valuable in that it may provide key associations between skills, which may be used to maximize the efficacy of training. For example, if two or more skills may be trained using the same training scenario, such a training scenario may be particularly useful for subject subpopulations which have difficulty with those two or more skills. As another example, if a first skill tends to improve when a second skill is trained, if a subject subpopulation struggles with the first skill and the second skill, it may be inferred that training the second skill with a particular training scenario may also benefit the first skill.

In step 309, the computing device 103 may associate skills together. Based on the skill association data 901 received in step 308, and/or based on the clustering in step 307, the computing device 103 may associate different skills together. In this manner, the computing device 103 might group skills based on data determined by the computing device 103 (e.g., the identified clusters in step 306) as well as external data (e.g., the skill association data 901 received in step 308). The different skills might be associated together using weights. For example, skills may be associated with one another with a weighting value such that, for example, a weighting value of 0.05 means that training one skill will barely affect the other, a weighting value of 1 indicates that training one skill will directly train the other, and a weighting value of 2.5 indicates that training one skill will significantly improve the other. Every skill need not be associated with another skill: for example, the clusters identified in step 306 and/or the skill association data 901 received in step 308 may indicate that certain skills have effectively no association with other skills (and, e.g., therefore have a weight of zero or a value very close to zero). As a particular example, a subject subpopulation is unlikely to improve their writing skills by practicing speaking loudly.

In step 310, the computing device may receive the confidence intervals 155 from databases such as the first database 129 and/or the second database 131. The confidence intervals 155 are one example of data which may be used during simulation of various clusters of subject subpopulations, training scenarios, and/or skills. In the case of the confidence intervals 155, these may be used to set confidence values for the quality and/or validity of the behavioral goal data 154 received in step 302, the skill data 153 received in step 302, the skill data 153 generated in step 304, the clusters of related skills identified in step 306, the skill association data 901 described in step 308, the associated skills (e.g., the weights) in step 309, or the like. In this manner, the confidence intervals 155 may indicate a degree with which certain aspects of data should be relied upon during simulation(s) of various clusters of subject subpopulations, training scenarios, and/or skills. Additionally and/or alternatively, the confidence intervals 155 may relate to the procedure with which simulations of such clusters are performed. For example, the confidence intervals 155 may be used in the process of simulation to distinguish between reliable simulations versus those which may be implausible.

In step 311, the computing device 103 may test various combinations of subject subpopulations, training scenarios, and/or skills. The computing device 103 may iteratively test various combinations of training scenarios and one or more subject subpopulations to determine how a simulated training scenario affects one or more skills of the one or more subject subpopulations. This step may thereby be similar to step 204 of FIG. 2. Such simulations may be performed with different combinations of subject subpopulations, different training scenarios (and/or combinations of training scenarios), or the like. For example, the computing device 103 may simulate the efficacy (e.g., with respect to skill improvement) of providing two different subject subpopulations a series of sequential training scenarios. The scenarios might be performed based on the confidence intervals received in step 310. For example, the computing device 103 might skip testing two training scenarios that have skills that have a de minimis association (e.g., a weight value that does not satisfy a predetermined threshold).

Like step 204 of FIG. 2, step 311 may entail iterating through various combinations of training scenarios and subject subpopulations to identify particularly effective combinations. Such combinations may be unintuitive, but may result from, for example, the associations identified in step 309. For example, it might not be obvious to a clinician that a certain training scenario may have beneficial knock-on effects for a wide variety of skills for a particular subject subpopulation, but the beneficial knock-on effects may nonetheless exist. By iteratively simulating these training scenarios in view of the commonly-unattained skills 701 identified in step 304, the clusters identified in step 306, and/or the associations determined in step 309, such knock-on effects may be identified and leveraged.

Moreover, like in step 205 of FIG. 2, step 311 may involve prioritizing combinations of subject subpopulations, training scenarios, and/or skills which are expected to yield maximum benefit and/or detection power. In other words, one of goal of the simulations performed in step 311 may be to identify unexpectedly efficacious combinations of one or more training scenarios and one or more subject subpopulations which maximally benefit one or more skills. That said, it is not necessary that a training scenario outright improve a skill for a particular subject subpopulation. In some circumstances, it may merely be beneficial for the combination to provide diagnostic information and/or allow a clinician to detect performance well.

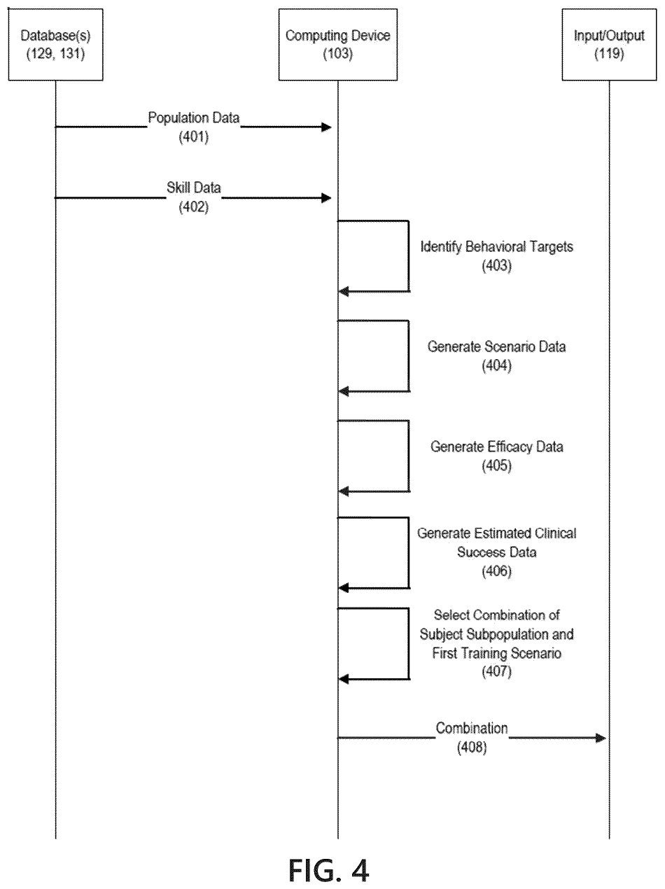

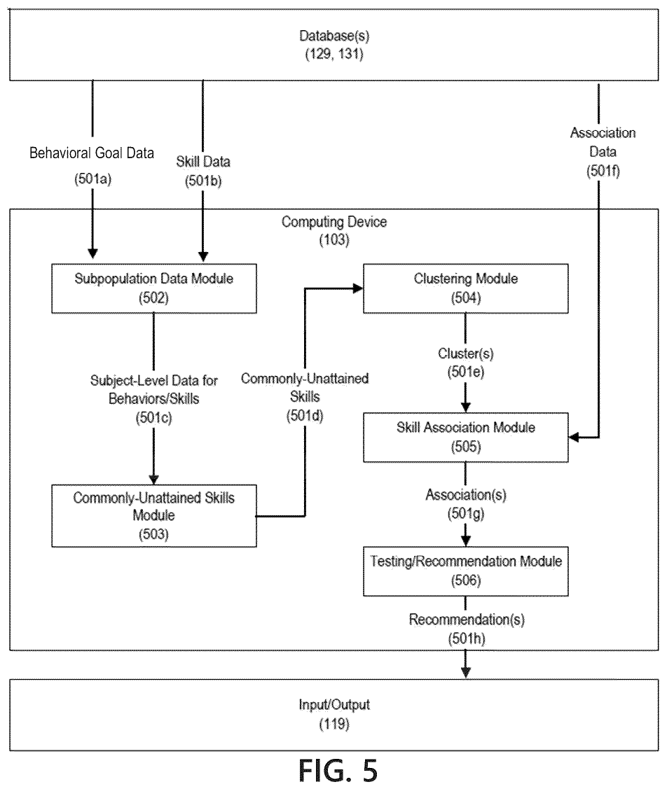

In step 312, the computing device may output (e.g., via the input/output 119) a recommendation. A recommendation (e.g., the recommendation 312 of FIG. 3, the combination 408 of FIG. 4, the recommendation(s) 501h of FIG. 5, or the like, as will be discussed in greater detail below) may be data related to one or more combinations of subject subpopulations, training scenarios, and/or skills as identified as part of step 311. For example, step 312 may involve outputting, to a clinician, an indication of a training scenario, which subject subpopulation(s) that should perform the training scenario, and the one or more skills that may be trained using the training scenario. The recommendation may be displayed in a user interface, such as in a user interface on a computing device associated with a clinician.

The recommendation may be configured to initiate one or more training scenarios. For example, the output indication may be configured to cause a computing device (e.g., a smartphone, a virtual reality headset) to begin a training scenario. This approach may be particularly useful where the training scenario can be performed using the computing device receiving the recommendation, such as may be the case for training scenarios involving practicing writing skills.

In particular, the recommendation (e.g., the recommendation 312 of FIG. 3, the combination 408 of FIG. 4, the recommendation(s) 501h of FIG. 5, or the like, as will be discussed in greater detail below) may cause an extended reality device (e.g., a virtual reality headset, an augmented reality headset, and/or a mixed reality headset) to provide, to a user associated with a subject subpopulation, an extended reality environment based on a training scenario. Training scenarios may be provided through a virtual reality, mixed reality, and/or augmented reality device. For example, a training scenario involving purchasing an item at a convenience store may be provided in virtual reality, rather than being provided in real life. As such, the recommendation may be configured to initiate the provision of the training scenario in a virtual reality, augmented reality, and/or mixed reality environment.

The recommendation may be transmitted to a user computing device, such that the user computing device may be caused to display an indication of the recommendation (e.g., the one or more combinations of subject subpopulations, training scenarios, and/or skills as identified as part of step 311). The recommendation may be provided to a clinician but may also additionally and/or alternatively be provided to another individual, such as a member of a subject subpopulation. For example, this may cause a subject's smartphone to output an indication that the subject should practice certain training scenarios.

FIG. 4 depicts a messaging diagram between the databases, the computing device 103, and input/output 119. The devices depicted in FIG. 4 are illustrative and may be rearranged as desired. For example, the databases may include, for example, the behavioral target database 141, the skill database 142, the scenario database 143, and/or the subject database 144. Like the devices, the messages depicted in FIG. 4 are also illustrative, and may be rearranged, omitted, and/or revised as desired.

In step 401, the computing device 103 may receive population data 151 from databases such as the first database 129, the second database 131, and/or the subject database 144. The population data 151 may indicate a plurality of different subject subpopulations. The population data 151 may be the same or similar as the population data 151 discussed above with respect to the subject database 144. The population data 151 may indicate, for example, various subjects, past diagnostics relating to those subjects, demographic information relating to those subjects, and the like. This step may be the same or similar as step 303 of FIG. 3.

In step 402, the computing device 103 may receive skill data 153. The skill data 153 may indicate a plurality of different skills associated with one or more behaviors exhibited by individuals experiencing one or more NDs. This step may be the same or similar as step 302 of FIG. 3.

In step 403, the computing device 103 may identify behavioral goals. The behavioral goals may be for each subject subpopulation of the plurality of different subject subpopulation. Such behavioral goals may be the same or similar as those discussed with respect to the behavioral target database 141 of FIG. 1. The behavioral goals may correspond to one or more commonly-unattained skills 701 for each subject subpopulation of the plurality of different subject subpopulation. In this manner, the behavioral goals may indicate one or more skills which are lacking in one or more subject subpopulations, such that those one or more skills are targets for improvement in a training setting. Accordingly, step 403 may be the same or similar as step 201 of FIG. 2 and/or step 301 of FIG. 3.

Identifying the behavioral goals may be based on data associated with particular subject subpopulations. Generally, one example of a behavioral goal is one or more commonly-unattained skills 701 for one or more subject subpopulations. As such, determining those commonly-unattained skills 701 may be based on diagnostic scores, such as a range of Full-Scale Intelligence Quotient (FSIQ) values, and/or a range of Social Responsiveness Scale—Version 2 (SRS Total) t-scores. In this manner, poor diagnostic scores may indicate that the skills are lacking. Moreover, determining such commonly-unattained skills 701 may also be based on a range of subject ages. After all, child subjects might not be expected to have the same competency at certain skills (e.g., personal hygiene, writing) as compared to adult subjects.

In step 404, the computing device 103 may generate scenario data 152. The scenario data 152 may indicate a plurality of different training scenarios for training one or more skills of the plurality of different skills. The scenario data 152 might be the same or similar as that discussed with respect to the scenario database 143. As such, generating the scenario data 152 may additionally and/or alternatively comprise retrieving the scenario data 152 from the scenario database 143. Moreover, step 404 may involve generating information about various training scenarios, such as those discussed with respect to step 201 of FIG. 2 and step 301 of FIG. 3.

In step 405, the computing device 103 may generate efficacy data. To generate the efficacy data, the computing device 103 may estimate, for each training scenario of the plurality of different training scenarios, a probability that each skill of the plurality of different skills can be trained by the training scenario. This step may be the same or similar as step 204 of FIG. 2 and/or step 308 through step 311 of FIG. 3.

In step 406, the computing device 103 may generate estimated clinical success data. To generate the estimated clinical success data, the computing device 103 may simulate, for each training scenario of the plurality of different training scenarios and for each subject subpopulation of the plurality of different subject subpopulation, a degree of behavioral change by the subject subpopulation. This step may be the same or similar as step 204 of FIG. 2 and/or step 311 of FIG. 3.

The estimated clinical success data may indicate mathematical calculations of an effect size on a performance level of a subject subpopulation. For example, the estimated clinical success data may indicate an absolute effect size of a performance level of the subject subpopulation and/or a standardized effect size of the performance level of the subject subpopulation. Such calculations may advantageously be used to aid in the ready comparison of various different simulated training scenarios. For example, by standardizing the effect size calculated for each simulated training scenario, the degrees of behavioral change indicated by such simulations may be more readily output.

Simulating the degree of behavioral change by the subject subpopulation may comprise use of various models. For example, simulating the degree of behavioral change by the subject subpopulation may comprise use of the Monte Carlo method to simulate a performance level of each subject subpopulation of the plurality of different subject subpopulations. Such models may be advantageous in that they may better estimate the outcome of training scenarios, particularly in circumstances where various random variables may be involved.

Simulating the degree of behavioral change may comprise weighting the performance level. It may be advantageous to weight the degree of the behavioral change such that it reflects a priority of the behavioral change: for example, the behavioral change may be particularly important in that it uniquely benefits a particularly rare subpopulation (e.g., one that is small or otherwise underserved) and/or particularly important if it pertains to critical skills (e.g., hygiene, which may negatively impact a subject's health if not addressed). To weight the degree of behavioral change, a function may be applied to a performance level of one or more subjects. That function may be based on a rarity of each subject subpopulation of the plurality of different subject subpopulations. In this manner, smaller and/or otherwise underserved subject subpopulations may be provided adequate representation in the data, rather than being effectively pushed out by larger/more noticeable subpopulations.

Generating the estimated clinical success data may be based on a standard. As such, the clinical success data may reflect expected performance (e.g., a degree of behavioral change) for one or more subjects and/or subject subpopulations with respect to established standards for one or more NDs. For example, generating the estimated clinical success data may comprise use of the Vineland Adaptive Behavior Scale (VABS), a Goal Attainment Scale (GAS), or a similar standard.

In step 407, the computing device 103 may select a combination of a subject subpopulation and a first training scenario. This selection process may be based on the behavioral goals, the efficacy data, and/or the estimated clinical success data. The first training scenario may be associated with training two or more of a plurality of different skills. This step may be the same or similar as step 205 of FIG. 2 and/or step 311 of FIG. 3.

Selecting the combination may comprise selecting at least two subject subpopulations of the plurality of different subject subpopulations. As indicated above with respect to FIGS. 2 and 3, multiple subject subpopulations may be trained using the same training scenario(s), and training scenario(s) may involve multiple subject subpopulations at the same time. For example, in a training scenario involving training subjects to practice speaking in social situations, women aged forty to fifty experiencing one or more NDs may be paired with men aged forty to fifty experiencing one or more NDs, such that the two subject subpopulations may assume different roles in the training scenario. This sort of combination reflects one of the many benefits of the system described herein: such unique combinations of subject subpopulations, training scenarios, and skills may be determined in manners which simply could not be determined by human review. Indeed, counterintuitive combinations may be identified by the computing device 103, and yet those counterintuitive combinations may be particularly valuable in helping train individuals experiencing one or more NDs.

Selecting the combination may comprise identifying that at least one of the different subject subpopulations has not performed a training scenario associated with the two or more of the plurality of different skills. In general, it may be desirable to select subpopulations for training scenarios such that the subpopulations learn skills. Indeed, it may be particularly useful to introduce subject subpopulations to training scenarios where they may improve multiple skills at the same time. In turn, selecting the combination may entail identifying that one or more subpopulations have not performed a training scenario associated with a plurality of different skills. After all, it may be wasteful to train subjects for a skill that have already trained.

Selecting the combination may be based on a trainability value (e.g., the trainability value(s) 156) assigned to the two or more of the plurality of different skills. For example, in some circumstances, a combination involving a training scenario that targets easily-trained skills may be selected, such that subjects are quickly provided success in their training program. In other circumstances, a combination involving a training scenario that targets more difficult-to-train skills may be selected, such that subjects can be helped in developing key skills.

In step 408, the computing device 103 may output (e.g., via the input/output 119) the combination. This step may be the same or similar as step 205 of FIG. 2 and/or step 312 of FIG. 3.

FIG. 5 represents another perspective of the message diagram of FIG. 3, in this instance focusing on various components of the computing device 103. In particular, the computing device 103 is shown with a subpopulation data module 502, a commonly-unattained skills module 503, a clustering module 504, a skill association module 505, and a testing/recommendation module 506.

In step 501a, behavioral goal data 154 may be received, by the subpopulation data module 502 of the computing device 103, from databases such as the first database 129, the second database 131, and/or the behavioral target database 141. This step may be the same or similar as step 301 of FIG. 3.

In step 501b, skill data 153 may be received, by the subpopulation data module 502 of the computing device 103, from databases such as the first database 129, the second database 131, and/or the skill database 142. This step may be the same or similar as step 302 of FIG. 3.

In step 501c, the subpopulation data module 502 of the computing device 103 may send subject-level data for behaviors/skills to the commonly-unattained skills module 503 of the computing device 103. This step may be the same or similar as step 303 of FIG. 3.

In step 501d, the commonly-unattained skills module 503 of the computing device 103 may send commonly-unattained skills (e.g., the commonly-unattained skills 701) to the clustering module 504 of the computing device 103. This step may be the same or similar as step 304 and/or step 305 of FIG. 3.

In step 501e, the clustering module 504 of the computing device 103 may send clusters of skills to the skill association module 505 of the computing device 103. This step may be the same or similar as step 306 and/or step 307 of FIG. 3.

In step 501f, one or more databases (e.g., the first database 129, the second database 131, and/or the skill database 142) may send skill association data (e.g., the skill association data 901) to the skill association module 505 of the computing device 103. This step may be the same or similar as step 308 of FIG. 3.

In step 501g, the skill association module 505 of the computing device 103 may send associations to the testing/recommendation module 506 of the computing device 103. This step may be the same or similar as step 309 through step 311 of FIG. 3.

In step 501h, the testing/recommendation module 506 of the computing device 103 may output (e.g., via the input/output 119) recommendations. This step may all or portions of step 312 of FIG. 3.

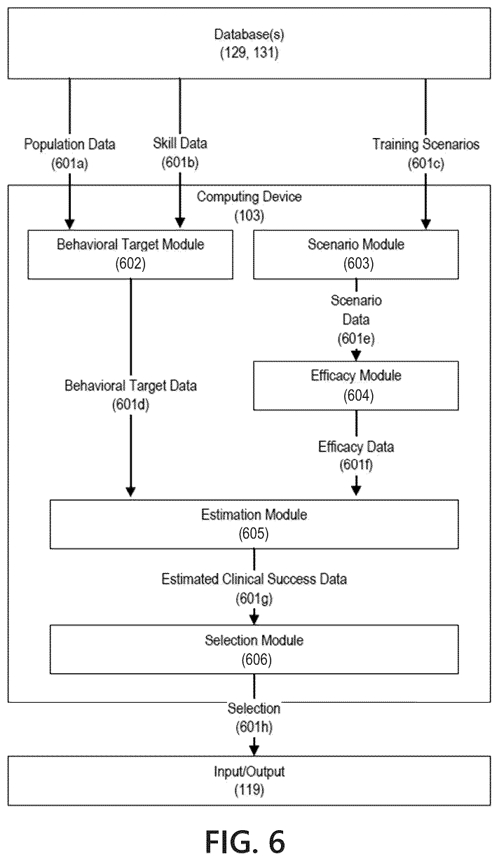

FIG. 6 represents another perspective of the message diagram of FIG. 4, in this instance focusing on various components of the computing device 103. In particular, the computing device 103 is shown with a behavioral goal module 602, a scenario module 603, an efficacy module 604, an estimation module 605, and a selection module 606.

In step 601a, the behavioral goal module 602 of the computing device 103 may receive population data 151 from databases such as the first database 129, the second database 131, and/or the subject database 144. This step may be the same or similar as step 401 of FIG. 4.

In step 601b, the behavioral goal module 602 of the computing device 103 may receive skill data 153 from databases such as the first database 129, the second database 131, and/or the skill database 142. This step may be the same or similar as step 402 of FIG. 4.

In step 601c, the scenario module 603 may receive training scenarios from databases such as the first database 129, the second database 131, and/or the scenario database 143. As indicated above with respect to FIG. 1, the scenario database 143, for example, may store information relating to training scenarios which may train one or more skills associated with behaviors exhibited by individuals experiencing one or more NDs. This step may be the same or similar as step 403 of FIG. 4.

In step 601d, the behavioral goal module 602 of the computing device 103 may send behavioral goal data 154 to the estimation module 605 of the computing device 103. This step may be the same or similar as step 404 of FIG. 4.

In step 601e, the scenario module 603 of the computing device 103 may send scenario data 152 to the efficacy module 604 of the computing device 103. This step may be the same or similar as step 405 of FIG. 4.

In step 601f, the efficacy module 604 of the computing device 103 may send efficacy data to the estimation module 605 of the computing device 103. This step may be the same or similar as step 406 of FIG. 4.

In step 601g, the estimation module 605 of the computing device 103 may send estimated clinical success data to the selection module 606 of the computing device 103. This step may be the same or similar as step 407 of FIG. 4.

In step 601h, the selection module 606 of the computing device 103 may output (e.g., via the input/output 119) a selection of, e.g., a combination of one or more subject subpopulations and/or one or more training scenarios. This step may be the same or similar as step 408 of FIG. 4.

FIG. 7 depicts an example of a chart depicting commonly-unattained skills 701 for a subject subpopulation. More particularly, this chart shows a percentage of twelve- to fifteen-year-olds experiencing one or more NDs that still fail to attain certain skills after simulated training scenarios are performed. This chart thereby illustrates one way in which the trainability of skills may be understood: broadly, while skills more towards the left of the x-axis are more trainable (e.g., can be more readily trained using training scenarios), skills more towards the right of the x-axis are less trainable (e.g., are more difficult to train using training scenarios).

The y-axis of the chart depicted in FIG. 7 recites one or more skills which may be associated with behaviors exhibited by individuals experiencing one or more NDs, such as ASD. These skills may correspond to items from the VABS. These skills are also grouped into various skill domains and skill subdomains. For example, the first two listed skills, “Notifies when Late or Absent” and “Uses Sav[ing]/Check[ing] [Account] Responsibly,” both correspond to the “Daily Living Skills” domain and the “Community” subdomain. As another example, the next two skills, “Goes Places Day Without Supervision” and “Plan Act[ivity] More than 2 [Things] Arranged,” correspond to the “Socialization” domain and the “Personal” subdomain. As yet another example, the fifth-listed skill, “Keeps Track of Medications,” corresponds to the “Daily Living Skills” domain and the “Personal” subdomain.

As suggested above, the x-axis of the chart depicted in FIG. 7 reflects the percentage of twelve- to fifteen-year-olds experiencing one or more NDs (e.g., ASD) that still fail to attain certain skills after simulated training scenarios are performed. In this case, lower values reflect more trainability (e.g., that training scenarios were more effective), and higher values reflect less trainability (e.g., that training scenarios were less effective). Thus, for example, it may be more difficult to train the skill “Cares for Minor Cuts” than it is to train “Notifies when Late or Absent.”

One benefit of aspects described herein is that the computing device 103 may discover strategies for training somewhat less trainable skills (e.g., “Cares for Minor Cuts,” “Has Conversations That Last 10 Minutes”) while training somewhat more easily trained skills (e.g., “Understands Sayings Not Word for Word,” “Writes Business Letters,” “Goes on Single Dates”).

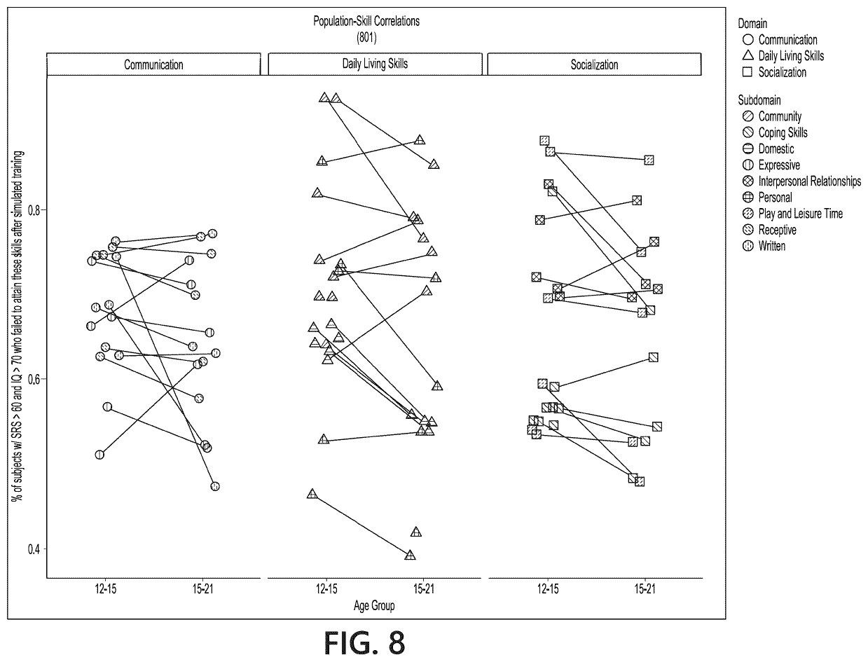

FIG. 8 depicts illustrative population-skill correlations 801 between different subpopulations and commonly-unattained skills (represented as points: circles for communication-domain skills, triangles for daily living skills, and squares for socialization skills). Per the population-skill correlations 801, in three domains (“Communication,” “Daily Living Skills,” “Socialization”), a percentage of subjects in two different subject subpopulations (12-15-year-olds and 15-21-year-olds) experiencing one or more NDs (e.g., ASD) who fail to attain these skills are depicted, with trajectory lines reflecting the improvement (or lack of improvement) in these skills. In other words, FIG. 8 may indicate whether subjects are likely to improve at performing certain skills as they age (as reflected by a negative trajectory line, which indicates a decreased failure rate in the subject subpopulation) or whether these subjects are likely to become worse at these skills as they age (as reflected by a positive trajectory line, which indicates an increased failure rate in the subject subpopulation). The computing device 103 may use the population-skill correlations 801, such as is depicted in FIG. 8, as part of, e.g., step 309 of FIG. 3. Indeed, the population-skill correlations depicted in FIG. 8 may represent all or portions of the skill association data (e.g., the skill association data 901) that the computing device 103 may receive as part of step 308 of FIG. 3.

One benefit of aspects described herein is that the population-skill correlations 801 may be used by the computing device 103 to better understand combinations of subject subpopulations and training scenarios which may be maximally beneficial for addressing behaviors associated with one or more NDs, such as ASD. For example, skills known to become worse over time (e.g., where the lines in FIG. 8 have a positive trajectory, indicating an increased failure rate), it may be beneficial to train both subject subpopulations (e.g., the 12-15-year-olds and the 15-21-year-olds) so as to proactively address the trend with respect to the 12-15-year-olds while also addressing the deficiency with respect to the 15-21-year-olds.

Another benefit of the population-skill correlations 801 depicted in FIG. 8 with respect to this disclosure is that certain skills may be associated with one another. For example, when comparing the trajectory lines between the 12-15-year-old subject subpopulation and the 15-21-year-old subject subpopulation, some skills appear to have similar failure rates and appear to have similar trajectory lines. These trends may suggest that the skills, although potentially not in the same subdomain (or even the same domain), may exhibit similarities and may warrant training together in the same training scenario. As such, the skill database 142 might store information such as data indicating the population-skill correlations 801.

FIG. 9 depicts simulated improvements, for a subject subpopulation, via one or more training scenarios. More particularly, FIG. 9 depicts skill association data 901 that reflects the benefits of the present disclosure: specifically, it shows the degree of benefit (as measured by a standard, here VABS) that might be recognized if particular subject subpopulations were provided one or more training scenarios. In this manner, FIG. 9 reflects data which may be part of, e.g., output from step 311 of FIG. 3 and/or step 406 of FIG. 4.

More specifically, FIG. 9 shows a predicted improvement to a VABS score by subjecting different subject subpopulations (3-year-olds and 7-year-olds) to one, two, three, four, or five training scenarios. The charts are further reflective of the IQ of the different subjects. Inspection of the charts suggests that, across both subject subpopulations, simulations indicate that increased numbers of training scenarios may improve subject subpopulation VABS scores. Moreover, the charts suggest that such improvements may be better recognized by the three-year-old subject subpopulation as compared to the seven-year-old subject subpopulation. Additionally, the charts suggest that the improvements are predicted to be better for subjects with a higher IQ; however, this trend is rather small.

In this manner, FIG. 9 is a window into the benefits of the present disclosure. Data such as that depicted in FIG. 9, which may be output as part of the present disclosure, provides critical insight into otherwise unknown dimensions of the world of training neurodevelopmental disorders such as ASD. In other words, clinicians or other humans could not process such information, whether mentally or with pen-and-paper: rather, this analysis is the result of the repeated and iterative simulation performed by the computing device 103.

FIG. 10 depicts an example of how a computing device, such as the computing device 103, may represent clusters of related skills 1001. More particularly, FIG. 10 shows clusters of skills that might not be attained for a subject subpopulation. Both the x andy axes of the output in FIG. 10 may represent different skills: in the case of FIG. 10, hundreds of different skills across different skill domains and skill subdomains. Within the intersections of the x and y axes of FIG. 10, a heat map is displayed representing associations between different skills. More specifically, for the purposes of illustration, in FIG. 10, darker colors represent greater correlations with nearby skills (e.g., a greater weight between associations of different skills), whereas lighter colors represent weaker correlations with nearby skills (e.g., a relatively weaker weight between associations of different skills). As a result, the output depicted in FIG. 10 shows a diagonal line where the same skills are compared (and thus the weight is at its highest possible value, as two identical skills are being compared).

As shown by the boxes drawn around certain portions of FIG. 10, certain skills might be correlated (e.g., might have associations between one another) even when the skills are not the same (or even in the same skill domain and/or skill subdomain). For example, while two skills might not be identical, they may nonetheless be related in that, for example, a positive effect on one might positively affect another. One particularly valuable aspect of the present disclosure is that it may be configured to detect associations between skills that, generally, would not be otherwise understood to be associated. Such activity is reflected in, e.g., step 306 of FIG. 3 and step 407 of FIG. 4. FIG. 10 reflects how such associations may be visualized in system output: specifically, it represents output (e.g., from the computing device 103) that indicates strong associations (e.g., strong weights) between different skills, including skills in different domains. In other words, FIG. 10 is an example of the unique way in which the computing device 103 may represent associations between various skills that may be targeted using aspects described herein.

One way in which output from the computing device 103, such as is represented in FIG. 10, might be used is to facilitate identification of potential training targets. The regions in FIG. 10 bounded by boxes may represent clusters where multiple skills (e.g., across various skill domains and/or skill subdomains) might be associated, such that training of one skill within that cluster might positively benefit other skills within that cluster. In turn, one or more training scenarios which target one or more skills in that cluster might be selected, such that other skills might be positively affected.

The following paragraphs (M1) through (M11) describe examples of methods that may be implemented in accordance with the present disclosure.

(M1) A method comprising receiving population data that indicates a plurality of different subject subpopulations; receiving skill data that indicates a plurality of different skills associated with one or more behaviors exhibited by individuals with one or more neurodevelopmental disorders; identifying, based on the population data and the skill data, behavioral goals for each subject subpopulation of the plurality of different subject subpopulation, wherein the behavioral goals correspond to one or more commonly-unattained skills for each subject subpopulation of the plurality of different subject subpopulation; generating scenario data which indicates a plurality of different training scenarios for training one or more skills of the plurality of different skills; generating efficacy data by estimating, for each training scenario of the plurality of different training scenarios, a probability that each skill of the plurality of different skills can be trained by the training scenario; generating estimated clinical success data by simulating, for each training scenario of the plurality of different training scenarios and for each subject subpopulation of the plurality of different subject subpopulation, a degree of behavioral change by the subject subpopulation; and selecting, based on the behavioral goals, the efficacy data, and the estimated clinical success data, a combination of a first subject subpopulation of the plurality of different subject subpopulations and a first training scenario of the plurality of different training scenarios, wherein the first training scenario of the plurality of different training scenarios is associated with training two or more of the plurality of different skills.

(M2) The method described in paragraph (M1), further comprising causing an extended reality device to provide, to a user associated with the first subject subpopulation, an extended reality environment based on the first training scenario.

(M3) The method described in any one of paragraphs (M1)-(M2), wherein selecting the combination comprises: identifying that at least one of the different subject subpopulations has not performed a training scenario associated with the two or more of the plurality of different skills.

(M4) The method described in any one of paragraphs (M1)-(M3), wherein generating the estimated clinical success data comprises using one or more of: the Vineland Adaptive Behavior Scale (VABS), or a Goal Attainment Scale (GAS).

(M5) The method described in any one of paragraphs (M1)-(M4), wherein selecting the combination is based on a trainability value assigned to the two or more of the plurality of different skills.

(M6) The method described in any one of paragraphs (M1)-(M5), wherein simulating the degree of behavioral change by the subject subpopulation comprises using the Monte Carlo method to simulate a performance level of each subject subpopulation of the plurality of different subject subpopulations.

(M7) The method described in any one of paragraphs (M1)-(M6), wherein selecting the combination comprises selecting at least two subject subpopulations of the plurality of different subject subpopulations.

(M8) The method described in any one of paragraphs (M1)-(M7), further comprising: transmitting, by the computing platform and to a user computing device, an indication of the combination, wherein transmitting the indication of the combination causes the user computing device to display the indication of the combination.

(M9) The method described in any one of paragraphs (M1)-(M8), wherein the estimated clinical success data indicates one or more of: an absolute effect size of a performance level of the subject subpopulation; or a standardized effect size of the performance level of the subject subpopulation.

(M10) The described in any one of paragraphs (M1)-(M9), wherein simulating the degree of behavioral change comprises weighting the performance level by applying a function to the performance level, wherein the function is based on a rarity of each subject subpopulation of the plurality of different subject subpopulations.

(M11) The method described in any one of paragraphs (M1)-(M10), wherein identifying the behavioral goals is based on one or more of: a range of subject ages; a range of Full-Scale Intelligence Quotient (FSIQ) values; or a range of Social Responsiveness Scale—Version 2 (“SRS Total”) t-scores.

The following paragraphs (A1) through (A11) describe examples of apparatuses that may be implemented in accordance with the present disclosure.

(A1) A computing device comprising: one or more processors; and memory storing instructions that, when executed by the one or more processors, cause the computing device to: receive population data that indicates a plurality of different subject subpopulations; receive skill data that indicates a plurality of different skills associated with one or more behaviors exhibited by individuals with one or more neurodevelopmental disorders; identify, based on the population data and the skill data, behavioral goals for each subject subpopulation of the plurality of different subject subpopulation, wherein the behavioral goals correspond to one or more commonly-unattained skills for each subject subpopulation of the plurality of different subject subpopulation; generate scenario data which indicates a plurality of different training scenarios for training one or more skills of the plurality of different skills; generate efficacy data by estimating, for each training scenario of the plurality of different training scenarios, a probability that each skill of the plurality of different skills can be trained by the training scenario; generate estimated clinical success data by simulating, for each training scenario of the plurality of different training scenarios and for each subject subpopulation of the plurality of different subject subpopulation, a degree of behavioral change by the subject subpopulation; and select, based on the behavioral goals, the efficacy data, and the estimated clinical success data, a combination of a first subject subpopulation of the plurality of different subject subpopulations and a first training scenario of the plurality of different training scenarios, wherein the first training scenario of the plurality of different training scenarios is associated with training two or more of the plurality of different skills.

(A2) The computing device described in paragraph (A1), wherein the instructions, when executed by the one or more processors, further cause the computing device to cause an extended reality device to provide, to a user associated with the first subject subpopulation, an extended reality environment based on the first training scenario.

(A3) The computing device described in any one of paragraphs (AT)-(A2), wherein the instructions, when executed by the one or more processors, further cause the computing device to select the combination by identifying that at least one of the different subject subpopulations has not performed a training scenario associated with the two or more of the plurality of different skills.

(A4) The computing device described in any one of paragraphs (AT)-(A3), wherein the instructions, when executed by the one or more processors, further cause the computing device to generate the estimated clinical success data using one or more of the Vineland Adaptive Behavior Scale (VABS), or a Goal Attainment Scale (GAS).

(A5) The computing device described in any one of paragraphs (A1)-(A4), wherein the instructions, when executed by the one or more processors, further cause the computing device to select the combination based on a trainability value assigned to the two or more of the plurality of different skills.

(A6) The computing device described in any one of paragraphs (A1)-(A5), wherein the instructions, when executed by the one or more processors, further cause the computing device to simulate the degree of behavioral change by the subject subpopulation using the Monte Carlo method to simulate a performance level of each subject subpopulation of the plurality of different subject subpopulations.

(A7) The computing device described in any one of paragraphs (A1)-(A6), wherein the instructions, when executed by the one or more processors, further cause the computing device to select the combination by selecting at least two subject subpopulations of the plurality of different subject subpopulations.

(A8) The computing device described in any one of paragraphs (A1)-(A7), wherein the instructions, when executed by the one or more processors, further cause the computing device to transmit by the computing platform and to a user computing device, an indication of the combination, wherein transmitting the indication of the combination causes the user computing device to display the indication of the combination.

(A9) The computing device described in any one of paragraphs (A1)-(A8), wherein the estimated clinical success data indicates one or more of: an absolute effect size of a performance level of the subject subpopulation; or a standardized effect size of the performance level of the subject subpopulation.

(A10) The computing device described in any one of paragraphs (A1)-(A9), wherein the instructions, when executed by the one or more processors, further cause the computing device to simulate the degree of behavioral change by weighting the performance level by applying a function to the performance level, wherein the function is based on a rarity of each subject subpopulation of the plurality of different subject subpopulations.

(A11) The computing device described in any one of paragraphs (A1)-(A10), wherein the instructions, when executed by the one or more processors, further cause the computing device to identify the behavioral goals is based on one or more of: a range of subject ages; a range of Full-Scale Intelligence Quotient (FSIQ) values; or a range of Social Responsiveness Scale—Version 2 (“SRS Total”) t-scores.

The following paragraphs (CRM1) through (CRM11) describe examples of computer-readable media that may be implemented in accordance with the present disclosure.

(CRM1) One or more non-transitory computer-readable media storing instructions that, when executed by one or more processors, cause a computing device to: receive population data that indicates a plurality of different subject subpopulations; receive skill data that indicates a plurality of different skills associated with one or more behaviors exhibited by individuals with one or more neurodevelopmental disorders; identify, based on the population data and the skill data, behavioral goals for each subject subpopulation of the plurality of different subject subpopulation, wherein the behavioral goals correspond to one or more commonly-unattained skills for each subject subpopulation of the plurality of different subject subpopulation; generate scenario data which indicates a plurality of different training scenarios for training one or more skills of the plurality of different skills; generate efficacy data by estimating, for each training scenario of the plurality of different training scenarios, a probability that each skill of the plurality of different skills can be trained by the training scenario; generate estimated clinical success data by simulating, for each training scenario of the plurality of different training scenarios and for each subject subpopulation of the plurality of different subject subpopulation, a degree of behavioral change by the subject subpopulation; and select, based on the behavioral goals, the efficacy data, and the estimated clinical success data, a combination of a first subject subpopulation of the plurality of different subject subpopulations and a first training scenario of the plurality of different training scenarios, wherein the first training scenario of the plurality of different training scenarios is associated with training two or more of the plurality of different skills.

(CRM2) The one or more non-transitory computer-readable media described in paragraph (CRM1), wherein the instructions, when executed by the one or more processors, further cause the computing device to cause an extended reality device to provide, to a user associated with the first subject subpopulation, an extended reality environment based on the first training scenario.

(CRM3) The one or more non-transitory computer-readable media described in any one of paragraphs (CRM1)-(CRM2), wherein the instructions, when executed by the one or more processors, further cause the computing device to select the combination by identifying that at least one of the different subject subpopulations has not performed a training scenario associated with the two or more of the plurality of different skills.

(CRM4) The one or more non-transitory computer-readable media described in any one of paragraphs (CRM1)-(CRM3), wherein the instructions, when executed by the one or more processors, further cause the computing device to generate the estimated clinical success data using one or more of: the Vineland Adaptive Behavior Scale (VABS), or a Goal Attainment Scale (GAS).

(CRM5) The one or more non-transitory computer-readable media described in any one of paragraphs (CRM1)-(CRM4), wherein the instructions, when executed by the one or more processors, further cause the computing device to select the combination based on a trainability value assigned to the two or more of the plurality of different skills.

(CRM6) The one or more non-transitory computer-readable media described in any one of paragraphs (CRM1)-(CRM5), wherein the instructions, when executed by the one or more processors, further cause the computing device to simulate the degree of behavioral change by the subject subpopulation using the Monte Carlo method to simulate a performance level of each subject subpopulation of the plurality of different subject subpopulations.

(CRM7) The one or more non-transitory computer-readable media described in any one of paragraphs (CRM1)-(CRM6), wherein the instructions, when executed by the one or more processors, further cause the computing device to select the combination by selecting at least two subject subpopulations of the plurality of different subject subpopulations.

(CRM8) The one or more non-transitory computer-readable media described in any one of paragraphs (CRM1)-(CRM7), wherein the instructions, when executed by the one or more processors, further cause the computing device to transmit by the computing platform and to a user computing device, an indication of the combination, wherein transmitting the indication of the combination causes the user computing device to display the indication of the combination.

(CRM9) The one or more non-transitory computer-readable media described in any one of paragraphs (CRM1)-(CRM8), wherein the estimated clinical success data indicates one or more of: an absolute effect size of a performance level of the subject subpopulation; or a standardized effect size of the performance level of the subject subpopulation.

(CRM10) The one or more non-transitory computer-readable media described in any one of paragraphs (CRM1)-(CRM9), wherein the instructions, when executed by the one or more processors, further cause the computing device to simulate the degree of behavioral change by weighting the performance level by applying a function to the performance level, wherein the function is based on a rarity of each subject subpopulation of the plurality of different subject subpopulations.

(CRM11) The one or more non-transitory computer-readable media described in any one of paragraphs (CRM1)-(CRM10), wherein the instructions, when executed by the one or more processors, further cause the computing device to identify the behavioral goals is based on one or more of: a range of subject ages; a range of Full-Scale Intelligence Quotient (FSIQ) values; or a range of Social Responsiveness Scale—Version 2 (“SRS Total”) t-scores.

Although the subject matter has been described in language specific to structural features and/or methodological acts, it is to be understood that the subject matter defined in the appended claims is not necessarily limited to the specific features or acts described above. Rather, the specific features and acts described above are described as example implementations of the following claims.

What is claimed is:

1. A method comprising:
at a computing platform comprising one or more processors and memory:
receiving population data that indicates a plurality of different subject subpopulations;
receiving skill data that indicates a plurality of different skills associated with one or more behaviors exhibited by individuals with one or more neurodevelopmental disorders;
identifying, based on the population data and the skill data, behavioral goals for each subject subpopulation of the plurality of different subject subpopulations, wherein the behavioral goals correspond to one or more commonly-unattained skills for each subject subpopulation of the plurality of different subject subpopulations;
generating scenario data which indicates a plurality of different training scenarios for training one or more skills of the plurality of different skills;
generating efficacy data by estimating, for each training scenario of the plurality of different training scenarios, a probability that each skill of the plurality of different skills can be trained by the training scenario;
generating estimated clinical success data by simulating, for each training scenario of the plurality of different training scenarios and for each subject subpopulation of the plurality of different subject subpopulations, a degree of behavioral change by the subject subpopulation; and
selecting, based on the behavioral goals, the efficacy data, and the estimated clinical success data, a combination of a first subject subpopulation of the plurality of different subject subpopulations and a first training scenario of the plurality of different training scenarios, wherein the first training scenario of the plurality of different training scenarios is associated with training two or more of the plurality of different skills.

2. The method of claim 1, further comprising:
causing an extended reality device to provide, to a user associated with the first subject subpopulation, an extended reality environment based on the first training scenario.

3. The method of claim 1, wherein selecting the combination comprises:
identifying that at least one of the different subject subpopulations has not performed a training scenario associated with the two or more of the plurality of different skills.

4. The method of claim 1, wherein generating the estimated clinical success data comprises using one or more of:
the Vineland Adaptive Behavior Scale (VABS), or
a Goal Attainment Scale (GAS).

5. The method of claim 1, wherein selecting the combination is based on a trainability value assigned to the two or more of the plurality of different skills.

6. The method of claim 1, wherein simulating the degree of behavioral change by the subject subpopulation comprises using the Monte Carlo method to simulate a performance level of each subject subpopulation of the plurality of different subject subpopulations.

7. The method of claim 1, wherein selecting the combination comprises selecting at least two subject subpopulations of the plurality of different subject subpopulations.

8. The method of claim 1, further comprising:
transmitting, by the computing platform and to a user computing device, an indication of the combination, wherein transmitting the indication of the combination causes the user computing device to display the indication of the combination.

9. The method of claim 1, wherein the estimated clinical success data indicates one or more of:
an absolute effect size of a performance level of the subject subpopulation; or
a standardized effect size of the performance level of the subject subpopulation.

10. The method of claim 1, wherein simulating the degree of behavioral change comprises weighting the performance level by applying a function to the performance level, wherein the function is based on a rarity of each subject subpopulation of the plurality of different subject subpopulations.

11. The method of claim 1, wherein identifying the behavioral goals is based on one or more of:
a range of subject ages;
a range of Full-Scale Intelligence Quotient (FSIQ) values; or
a range of Social Responsiveness Scale—Version 2 (“SRS Total”) t-scores.

12. A computing device comprising:
one or more processors; and
memory storing instructions that, when executed by the one or more processors, cause the computing device to:
receive population data that indicates a plurality of different subject subpopulations;
receive skill data that indicates a plurality of different skills associated with one or more behaviors exhibited by individuals with one or more neurodevelopmental disorders;
identify, based on the population data and the skill data, behavioral goals for each subject subpopulation of the plurality of different subject subpopulations, wherein the behavioral goals correspond to one or more commonly-unattained skills for each subject subpopulation of the plurality of different subject subpopulations;
generate scenario data which indicates a plurality of different training scenarios for training one or more skills of the plurality of different skills;
generate efficacy data by estimating, for each training scenario of the plurality of different training scenarios, a probability that each skill of the plurality of different skills can be trained by the training scenario;
generate estimated clinical success data by simulating, for each training scenario of the plurality of different training scenarios and for each subject subpopulation of the plurality of different subject subpopulations, a degree of behavioral change by the subject subpopulation; and
select, based on the behavioral goals, the efficacy data, and the estimated clinical success data, a combination of a first subject subpopulation of the plurality of different subject subpopulations and a first training scenario of the plurality of different training scenarios, wherein the first training scenario of the plurality of different training scenarios is associated with training two or more of the plurality of different skills.

13. The computing device of claim 12, wherein the instructions, when executed by the one or more processors, cause the computing device to:
cause an extended reality device to provide, to a user associated with the first subject subpopulation, an extended reality environment based on the first training scenario.

14. The computing device of claim 12, wherein the instructions, when executed by the one or more processors, cause the computing device to select the combination by causing the computing device to:
identify that at least one of the different subject subpopulations has not performed a training scenario associated with the two or more of the plurality of different skills.

15. The computing device of claim 12, wherein the instructions, when executed by the one or more processors, cause the computing device to generate the estimated clinical success data comprises using one or more of:
the Vineland Adaptive Behavior Scale (VABS), or
a Goal Attainment Scale (GAS).

16. The computing device of claim 12, wherein the instructions, when executed by the one or more processors, cause the computing device to select the combination based on a trainability value assigned to the two or more of the plurality of different skills.

17. The computing device of claim 12, wherein the instructions, when executed by the one or more processors, cause the computing device to simulate the degree of behavioral change by the subject subpopulation using the Monte Carlo method to simulate a performance level of each subject subpopulation of the plurality of different subject subpopulations.

18. The computing device of claim 12, wherein the instructions, when executed by the one or more processors, cause the computing device to select the combination by causing the computing device to select at least two subject subpopulations of the plurality of different subject subpopulations.

19. One or more non-transitory computer-readable media storing instructions that, when executed by one or more processors, cause a computing device to:
receive population data that indicates a plurality of different subject subpopulations;
receive skill data that indicates a plurality of different skills associated with one or more behaviors exhibited by individuals with one or more neurodevelopmental disorders;
identify, based on the population data and the skill data, behavioral goals for each subject subpopulation of the plurality of different subject subpopulations, wherein the behavioral goals correspond to one or more commonly-unattained skills for each subject subpopulation of the plurality of different subject subpopulations;
generate scenario data which indicates a plurality of different training scenarios for training one or more skills of the plurality of different skills;
generate efficacy data by estimating, for each training scenario of the plurality of different training scenarios, a probability that each skill of the plurality of different skills can be trained by the training scenario;
generate estimated clinical success data by simulating, for each training scenario of the plurality of different training scenarios and for each subject subpopulation of the plurality of different subject subpopulations, a degree of behavioral change by the subject subpopulation; and
select, based on the behavioral goals, the efficacy data, and the estimated clinical success data, a combination of a first subject subpopulation of the plurality of different subject subpopulations and a first training scenario of the plurality of different training scenarios, wherein the first training scenario of the plurality of different training scenarios is associated with training two or more of the plurality of different skills.

20. The one or more non-transitory computer-readable media of claim 19, wherein the instructions, when executed by the one or more processors, cause the computing device to:
cause an extended reality device to provide, to a user associated with the first subject subpopulation, an extended reality environment based on the first training scenario.
